(19) United States
(12) Patent Application Publication
(10) Pub. No.: US 2025/0177146 A1
(43) Pub. Date: Jun. 5, 2025

(54) CRANIAL FIXATION DEVICE FOR DECOMPRESSIVE / AUGMENTATIVE CRANIECTOMY

(71) Applicant: NTPLAST S.R.L., Roma (IT)

(72) Inventor: Luigi BERRA, Roma (IT)

(73) Assignee: NTPLAST S.R.L., Roma (IT)

(21) Appl. No.: 18/842,176

(22) PCT Filed: Feb. 22, 2023
(86) PCT No.: PCT/IT2023/050059

§ 371 Date: Aug. 28, 2024

(30) Foreign Application Priority Data
Mar. 2, 2022 (IT) 102022000003821

(51) Int. Cl. A61F 2/28; A61B 17/68

(52) U.S. Cl. CPC A61F 2/2875; A61B 17/688

(57) ABSTRACT

A cranial fixation device for decompressive/augmentative craniectomy is described comprising a lid (4) and a plurality of brackets (3), the device being designed for the treatment of pathologies causing a herniation of the brain with a consequent increase in intracranial pressure, of the type which provides for the creation of an opening in the cranial case, by milling along a craniotomy edge (2), able to allow the expansion of the brain and the application of the lid (4) at a certain vertical distance (d) from said craniotomy rim (2) with functions of protection and containment of the brain, wherein the lid (4) is connected to the craniotomy rim (2) by the plurality of brackets (3) which are fixed simultaneously to the lid (4) and to the craniotomy rim (2).

The present invention relates to a cranial fixation device for decompressive/augmentative craniectomy.

The braincase is a rigid and non-expandable envelope and the increase in volume of its contents due to the presence of a brain tumour, an infection, a haemorrhage, a hydrocephalus, causes an increase in intracranial pressure. The increase in intracranial pressure beyond certain limits is a harmful and potentially fatal condition, capable of triggering cascade phenomena that result in ischemia and cerebral necrosis.

The treatment of intracranial hypertension involves the use of certain drugs and sedation and ventilation techniques, as well as the removal of CSF or space-occupying lesions (tumours, hematomas). In many cases, however, this is not sufficient to bring the intracranial pressure back to acceptable values and it is necessary to resort to decompressive craniectomy. This technique involves removing a large portion of the skull cap and opening the dura mater, thus allowing the swollen brain to herniate outward through the surgical skull defect. Decompressive craniectomy improves the clinical outcome in many situations, through different mechanisms: it reduces intracranial pressure, improves cerebral perfusion and reduces the risk of cerebral herniations and the consequent compression of vital structures such as the brainstem. Patients who survive the acute phase of intracranial hypertension will then undergo a cranioplasty operation which consists in the repositioning of the previously removed bone operculum or in the positioning of a skull case prosthesis made of synthetic material.

The period between the decompressive craniectomy and the subsequent cranioplasty, of the order of weeks or months, exposes the brain to the risk of damage from trauma for which it is necessary for the patient to use protections such as the helmets. Then, there are other complications linked to the absence of the bone operculum such as the “trephined syndrome”, caused by the effects of atmospheric pressure which are transmitted directly to the brain tissue, and those linked to the adhesions which are created between the subcutaneous soft tissues and the dura mater and the brain, with the possibility of bleeding and increased operating times and surgical risks in the bone repositioning phase.

In order to reduce the risks deriving from the absence of the bone operculum and from the cranioplasty operation, alternative solutions to the classic decompressive craniectomy have been devised. These alternative techniques have in common the fact that the bone operculum, after being cut and separated from the remaining cranial case, is left in place.

These techniques are called “hinged craniectomy”, when the bony operculum is fixed only on one side so as to be hinged and open like a door under the pressure of cerebral edema (see for example patent U.S. Pat. No. 8,439,956B2 by Kathryn Ko) or “floating craniectomy”, when the bone flap is left free to float on the herniated brain.

These techniques require precautions to avoid sinking of the bone once the cerebral edema and the consequent centrifugal thrust have ceased.

There are also telescopic fixing systems that allow the lifting and subsequent return of the bone operculum towards the original position once the outward thrust of the brain tissue has ceased (of this type there are several patents by Khanna, e. g. U.S. Pat. No. 9,603,626B2).

In the systems described above (hinged, telescopic or floating), the increase in the intracranial volume, which takes the form of herniation of the brain tissue through the craniotomy breach, occurs due to the thrust of the brain on the bone itself. The brain must therefore overcome the resistance, by no means modest, of the skin and muscle flap. In the case of hinged craniectomies then, the thrust must overcome the resistance of the muscle-skin flap through the bone which can only be separated on one side. In all cases, part of the volume that could be used for brain expansion is occupied by the volume of the bony lid itself, potentially reducing the effectiveness of the intervention.

Object of the present invention is overcoming the disadvantage complained of through the use of a device which allows the bone operculum to be left in place but, unlike the classic technique of decompressive craniectomy, is based on a completely different concept from that of the other systems, which could be defined as augmentative craniectomy, which instead involves expanding the skull in a stable manner.

The one described and other objects, as will be explained hereinafter, are achieved with the use of a cranial fixation device for decompressive/augmentative craniectomy according to claim 1, which allows the brain to herniate freely, while providing at the same time an effective protection to the brain itself.

Said device, suitable for the treatment of pathologies that cause a herniation of the brain with a consequent increase in intracranial pressure, is of the type which provides for:
the creation of an opening in the cranial case, by milling along a craniotomy edge, capable of allowing the expansion of the brain;
the application of a cover with functions of protection and containment of said brain;
wherein said lid is connected to the craniotomy rim by a plurality of brackets which are simultaneously secured to the lid and to the craniotomy rim.

The length of these stapes determines the increase in volume available to the herniated brain.

According to a preferred embodiment, said lid can be made of biocompatible synthetic material or the bone operculum itself taken by means of a craniotomy. In this case, the bone milling must take place following a cutting guide in order to have a coherence between the opening and the synthetic lid.

The lid is then kept raised by the brackets, overcoming the resistance of the musculocutaneous flap. These brackets have the characteristic of being sufficiently resistant to guarantee a seal even in the event of trauma to the head and of being sufficiently malleable with special pliers so as to be able to adapt at the time of implantation to the lid in synthetic material or to the bone operculum to be lifted.

This system is based on the observation of numerous patients undergoing classic decompressive craniectomy. The analysis of the radiological images made it possible to quantify the degree of herniation and this formed the basis for determining the volume that the device according to the invention must leave available to the brain to allow free herniation.

Since cerebral edema and intracranial hypertension are dynamic processes, it was possible to evaluate and quantify the maximum degree of expansion, allowing the height of the brackets to be designed accordingly.

The device according to the invention makes it possible to provide the brain with the space it needs to herniate and therefore to allow it to expand freely within this space.

According to a preferred embodiment, said brackets are made of titanium and have a central body, with the function of spacer, and two brackets which stabilize them in the anchorage point on the skull and prevent sinking of the bone operculum.

Preferred embodiments and non-trivial variants of the present invention form the subject of the dependent claims.

It is understood that all attached claims form an integral part of the present description.

The present invention solves the drawbacks in question since the brain is completely free to herniate, without having to overcome resistance of any kind for moving the operculum, since said operculum is already positioned so as to provide the brain with all the space it needs.

It will be immediately obvious that innumerable variations and modifications can be made to what is described (for example relating to shape, dimensions, arrangements and parts with equivalent functionality) without departing from the scope of the invention, as appears from the attached claims.

The present invention will be better described by a preferred embodiment thereof, provided by way of non-limiting example, with reference to the attached drawings, in which:

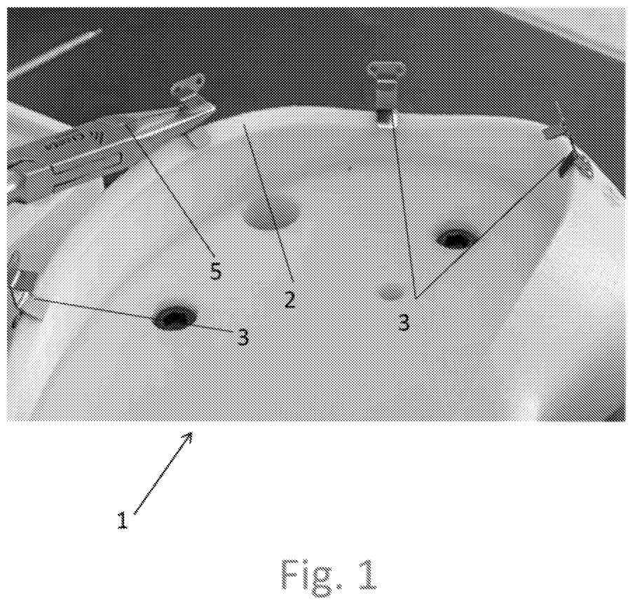

FIG. 1 shows a simulacrum of the skull case;

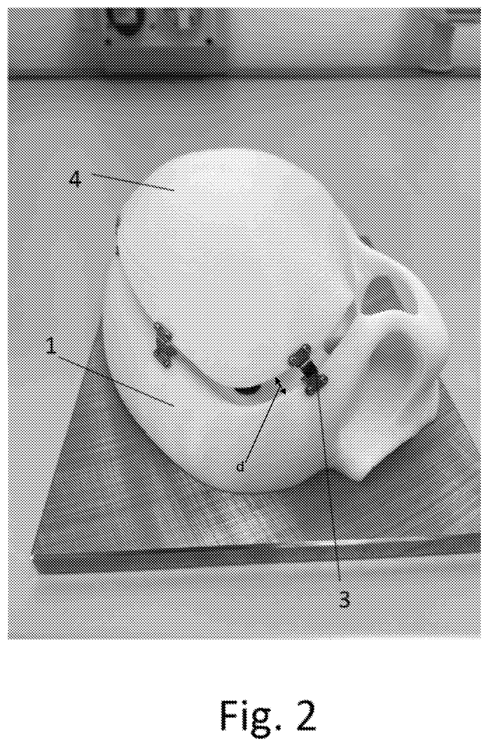

FIG. 2 shows the device according to the invention with the operculum held in position by a plurality of brackets;

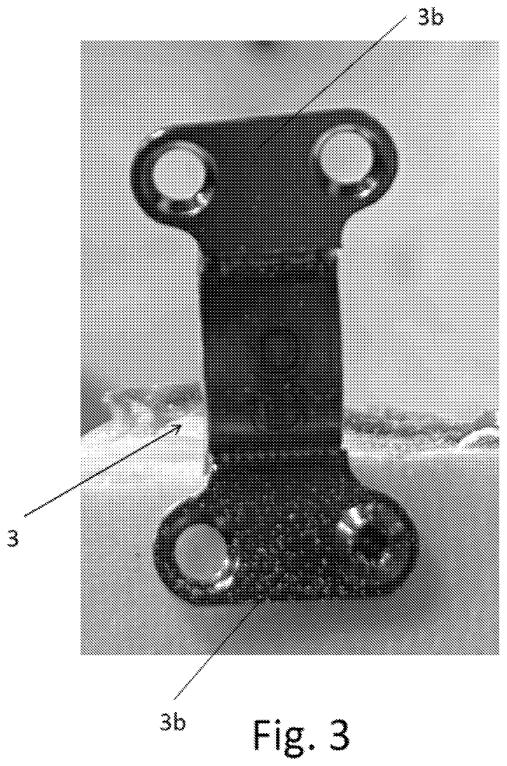

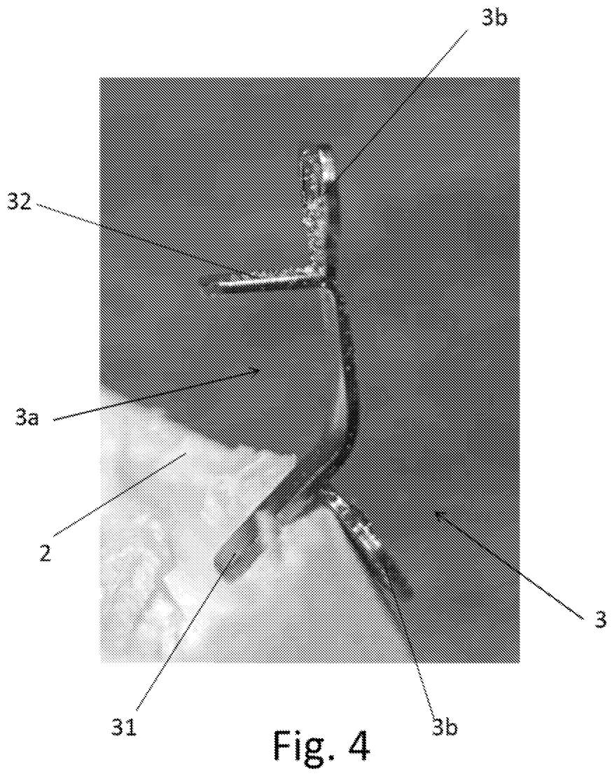

FIGS. 3 and 4 show a bracket for fixing said lid.

With reference to the attached figures, (1) indicates a simulacrum of a human skull, on which an opening has been made by milling along a craniotomy edge (2). A plurality of brackets (3) are fixed along said edge (2) by means of screws (not referenced) and a cover (4) is fixed on said brackets (3).

The brackets (3) are fixed to the skull (1) and to the lid (4) by means of screws.

These brackets (3) are made of titanium and include a central body (3a), with two shelves (31) and (32), which act as spacers, and two further shelves (3b) which fix them at the anchor point on the skull and prevent the cover from sinking (4).

In FIG. 4, the bracket (3) is shown mounted on the craniotomy edge (2) and it is highlighted how the lower shelf (31) of the central body (3a) rests on the craniotomy edge (2), while the upper shelf (32) is adapted to receive the cover (4). The position of the cover (4) is then defined by the length of the central part (3a) of the bracket (3).

The brackets (3) are strong enough to guarantee a seal even in the event of trauma to the head and malleable enough with special pliers (5) so as to be able to adapt when the lid is implanted (4).

According to another preferred embodiment, the brackets (3) are made of thermoplastic material (for example polyether ether ketone—PEEK) or in composite material with a thermoplastic matrix (for example carbon fibres impregnated with PEEK or other thermoplastic polymer). In this case, to shape the brackets (3) it is necessary to heat them adequately.

According to a further preferred embodiment, said lid (4) is the same bone coming from the craniotomy. It can be made in other ways, for example by 3D printing in a biocompatible synthetic material, for example polymethyl methacrylate (PMMA)

FIG. 1 highlights the part of the braincase on which the opening that allows the brain to herniate has been made, while FIG. 2 shows the skull with the opening to which the lid (4) has been applied, with a protective function of the brain but in a suitable position to provide the brain itself with the space it needs to herniate without exerting any effort.

As is clear from the preceding description, the fundamental technical problem faced has been completely solved, since the device according to the invention defines a space for free herniation of the brain.

In summary, the device of the invention serves for a cranial fixation for decompressive/augmentative craniectomy and comprises a lid (4) and a plurality of brackets (3), the device being designed for the treatment of pathologies which cause a herniation of the brain with a consequent increase in intracranial pressure, of the type which provides for the creation of an opening in the cranial case, by means of milling along a craniotomy edge (2), able to allow the expansion of the brain and the application of the lid (4) at a certain distance (d) vertically from the craniotomy edge (2) with functions of protection and containment of the brain, wherein the lid (4) is connected to the craniotomy edge (2) by the plurality of brackets (3) which are fixed simultaneously to the lid (4) and to the craniotomy edge (2), the brackets (3) comprising:
a central body (3a), having the function of spacer between the craniotomy edge (2) and the lid (4); and
two shelves (3b) which fix the brackets (3) in the anchorage point on the skull and prevent the lid from sinking (4).

Preferably, in the device indicated above, the lid (4) is made of synthetic material, in particular polymethyl methacrylate (PMMA).

Preferably, the lid (4) is made by 3D printing.

As a variant, in the device of the invention, the lid (4) can be the same bone operculum taken by means of a craniotomy.

The brackets (3) are strong enough to guarantee a seal even in the event of trauma to the head and are malleable enough with special pliers (5) so as to be able to adapt when the lid is implanted (4); in particular, the brackets (3) are made of titanium, or of thermoplastic material, again in particular this thermoplastic material is polyether ether ketone (PEEK) or a composite comprising carbon fibres impregnated with PEEK.

1. Cranial fixation device for decompressive/augmentative craniectomy comprising a lid (4) and a plurality of brackets (3), said device being designed for the treatment of pathologies causing herniation of the brain with a consequent increase in intracranial pressure, of the type which provides for the creation of an opening in the cranial case, by milling along a craniotomy edge (2), able to allow the expansion of the brain and the application of said lid (4) to a certain vertical distance (d) from said craniotomy edge (2) with functions of protection and containment of the brain, characterized in that said lid (4) is connected to the craniotomy edge (2) by said plurality of brackets (3) which are fixed simultaneously to the lid (4) and to the craniotomy edge (2), said brackets (3) comprising:
a central body (3a), having the function of spacer between the craniotomy edge (2) and the lid (4); and
two shelves (3b) which fix said brackets (3) in the anchorage point on the skull and prevent the lid (4) from sinking.

2. Cranial fixation device for decompressive/augmentative craniectomy according to claim 1, characterized in that said cover (4) is made of synthetic material.

3. Cranial fixation device for decompressive/augmentative craniectomy according to claim 2, characterized in that said synthetic material is polymethylmethacrylate (PMMA).

4. Cranial fixation device for decompressive/augmentative craniectomy according to claim 2, characterized in that said lid (4) is made by 3D printing.

5. Cranial fixation device for decompressive/augmentative craniectomy according to claim 1, characterized in that said lid (4) is the same bone operculum taken by means of a craniotomy.

6. Cranial fixation device for decompressive/augmentative craniectomy according to claim 1, characterized in that said brackets (3) are strong enough to guarantee a seal even in the event of trauma to the head and sufficiently malleable with special pliers (5) so that it can adapt when the lid (4) is implanted.

7. Cranial fixation device for decompressive/augmentative craniectomy according to claim 6, characterized in that said brackets (3) are made of titanium.

8. Cranial fixation device for decompressive/augmentative craniectomy according to claim 6, characterized in that said brackets (3) are made of thermoplastic material.

9. Cranial fixation device for decompressive augmentative craniectomy according to claim 8, characterized in that said thermoplastic material is polyether ether ketone (PEEK) or a composite comprising carbon fibres impregnated with PEEK.
